(19) United States
(12) Patent Application Publication
(10) Pub. No.: US 2019/0269829 A1
(43) Pub. Date: Sep. 5, 2019

(54) BONE GROWTH FACILITATION DEVICE AND METHODS OF USE

(71) Applicants: Bruce HOFER, Sunbury, OH (US); NANOFIBER SOLUTIONS, LLC, Hilliard, OH (US)

(72) Inventors: Jed JOHNSON, London, OH (US); Bruce HOFER, Sunbury, OH (US); Devan OHST, Columbus, OH (US)

(21) Appl. No.: 16/292,026

(22) Filed: Mar. 4, 2019

Related U.S. Application Data
(60) Provisional application No. 62/637,649, filed on Mar. 2, 2018.

(51) Int. Cl. A61L 27/44; A61F 2/28; A61L 27/56; A61L 27/54

(52) U.S. Cl. CPC A61L 27/446; A61F 2/28; A61L 27/56; A61F 2002/30784; A61F 2002/2835; A61F 2310/00329; A61F 2310/00359; A61L 27/54

(57) ABSTRACT

The instant disclosure is directed to devices and methods for facilitating bone growth. In one embodiment, a device may include a porous flexible tube comprising an electrospun fiber. The porous flexible tube may also comprise a closed end. The device may further comprise a filler material at least partially encased by the porous flexible tube. A method of manufacturing such a device may comprise electrospinning a polymer solution onto an end of a cylindrical mandrel to form the porous flexible tube, and removing the porous flexible tube from the end of the cylindrical mandrel. A method of facilitating bone growth may comprise obtaining such a device, and implanting the porous flexible tube into a subject's bone defect.

### CROSS-REFERENCE TO RELATED APPLICATIONS

This application claims priority to and benefit of U.S. Provisional Application Ser. No. 62/637,649, filed Mar. 2, 2018, entitled “Bone Growth Facilitation Device and Methods of Use,” which is incorporated herein by reference in its entirety.

### BACKGROUND

Surgical reconstruction of bone defects is becoming increasingly common. Spinal fusion, spinal reconstruction, tooth loss, periodontal disease, infections, trauma, congenital defects, tumors, malignant diseases such as cancer, and a multitude of other conditions may necessitate such reconstruction. Some surgical techniques seek to encourage new bone growth (“osteogenesis”) and bone reconstruction. Encouraging osteogenesis may help provide a fixation point for a permanent implant, or it may be necessary for a defect that is too large to repair with fixation alone. Bone growth facilitation may be particularly important in the context of spinal surgery, where bone grafting can allow osteogenesis to occur across a gap that could otherwise not be bridged by new bone. However, many bone graft materials are difficult to localize to a particular surgical site where they are most needed. Furthermore, large defects may require additional materials to facilitate bone healing. Therefore, there exists a need for devices and methods to facilitate bone growth in the context of surgical repair of bone defects.

### SUMMARY

The instant disclosure is directed to devices and methods for facilitating bone growth. In one embodiment, a device for facilitating bone growth may comprise a porous flexible tube comprising an electrospun fiber. In some embodiments, the porous flexible tube may comprise a closed end. In certain embodiments, the device may further comprise a filler material at least partially encased by the porous flexible tube. In other embodiments, the electrospun polymer fiber of the device may comprise a bioresorbable polymer. In still other embodiments, the porous flexible tube may have two closed ends.

In another embodiment, a method of manufacturing a device for facilitating bone growth may comprise electrospinning a polymer solution onto an end of a cylindrical mandrel to form a porous flexible tube comprising an electrospun polymer fiber, and removing the porous flexible tube from the end of the cylindrical mandrel. In some embodiments, the porous flexible tube may comprise a closed end. In certain embodiments, the method may further comprise at least partially encasing a filler material within the porous flexible tube. In other embodiments, the method may further comprise adding pores to the porous flexible tube using a laser ablation technique.

In yet another embodiment, a method of facilitating bone growth may comprise obtaining a porous flexible tube comprising an electrospun polymer fiber, and implanting the porous flexible tube into a subject's bone defect. In some embodiments, the porous flexible tube may comprise a closed end. In certain embodiments, the method may further comprise at least partially encasing a filler material within the porous flexible tube. Further embodiments of the instant disclosure are described herein.

### BRIEF DESCRIPTION OF THE DRAWINGS

FIG. 1 illustrates embodiments of a porous flexible tube (bottom) and a device comprising a filler material at least partially encased by the porous flexible tube (top), in accordance with the present disclosure.

FIG. 2A illustrates a histological sample of a native ovine rotator cuff.

FIG. 2B illustrates a histological sample of an ovine rotator cuff, at 12 weeks, repaired without using electrospun polymer fibers.

FIG. 2C illustrates a magnified view of a portion of the histological sample shown in FIG. 2B.

FIG. 2D illustrates a histological sample of an ovine rotator cuff, at 12 weeks, repaired with electrospun polymer fibers in accordance with the instant disclosure.

FIG. 2E illustrates a magnified view of a portion of the histological sample shown in FIG. 2D.

FIG. 3A illustrates repair of defects on the distal surface of a rabbit femur using Cartiform® Viable Osteochondral Allograft in which one side of a bilateral 4.75 mm diameter approximately 4 mm deep defect was repaired with Cartiform®.

FIG. 3B illustrates repair of defects on the distal surface of a rabbit femur using a scaffold as described in embodiments herein (a “Nanofiber Solutions” scaffold), in which one side of a bilateral 4.75 mm diameter approximately 4 mm deep defect was repaired with a nanofiber scaffold in accordance with the instant disclosure.

FIG. 4A illustrates a histological sample of the femur repaired using Cartiform®.

FIG. 4B illustrates a histological sample of the femur repaired using the Nanofber Solutions scaffold in accordance with the instant disclosure.

### DETAILED DESCRIPTION

This disclosure is not limited to the particular systems, devices and methods described, as these may vary. The terminology used in the description is for the purpose of describing the particular versions or embodiments only, and is not intended to limit the scope of the disclosure.

The following terms shall have, for the purposes of this application, the respective meanings set forth below. Unless otherwise defined, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

As used herein, the singular forms “a,” “an,” and “the” include plural references, unless the context clearly dictates otherwise. Thus, for example, reference to a “fiber” is a reference to one or more fibers and equivalents thereof known to those skilled in the art, and so forth.

As used herein, the term “about” means plus or minus 10% of the numerical value of the number with which it is being used. Therefore, about 50 mm means in the range of 45 mm to 55 mm.

As used herein, the term “consists of” or “consisting of” means that the device or method includes only the elements, steps, or ingredients specifically recited in the particular claimed embodiment or claim.

In embodiments or claims where the term comprising is used as the transition phrase, such embodiments can also be envisioned with replacement of the term “comprising” with the terms “consisting of” or “consisting essentially of.”

The terms “animal,” “patient,” and “subject” as used herein include, but are not limited to, humans and non-human vertebrates such as wild, domestic, and farm animals. In some embodiments, the terms “animal,” “patient,” and “subject” may refer to humans.

As used herein, the term “biocompatible” refers to non-harmful compatibility with living tissue. Biocompatibility is a broad term that describes a number of materials, including bioinert materials, bioactive materials, bioabsorbable materials, biostable materials, biotolerant materials, or any combination thereof.

As used herein, the terms “tube” and “flexible tube” describe scaffolds are not limited to perfectly or uniformly tubular or cylindrical shapes. A flexible tube may encompass, for example, an oval shape, a sheet rolled to form a tube, a sheet folded to create a pocket, and other possible configurations of a scaffold to form an internal space. A flexible tube may be capable of being formed into a tubular or cylindrical shape, but need not hold its shape without a filler material, an external structure, or a combination thereof.

### Electrospinning Fibers

Electrospinning is a method which may be used to process a polymer solution into a fiber. In embodiments wherein the diameter of the resulting fiber is on the nanometer scale, the fiber may be referred to as a nanofiber. Fibers may be formed into a variety of shapes by using a range of receiving surfaces, such as mandrels or collectors. In some embodiments, a flat shape, such as a sheet or sheet-like fiber mold, a fiber scaffold and/or tube, or a tubular lattice, may be formed by using a substantially round or cylindrical mandrel. In certain embodiments, the electrospun fibers may be cut and/or unrolled from the mandrel as a fiber mold to form the sheet. The resulting fiber molds or shapes may be used in many applications, including the repair or replacement of biological structures. In some embodiments, the resulting fiber scaffold may be implanted into a biological organism or a portion thereof.

Electrospinning methods may involve spinning a fiber from a polymer solution by applying a high DC voltage potential between a polymer injection system and a mandrel. In some embodiments, one or more charges may be applied to one or more components of an electrospinning system. In some embodiments, a charge may be applied to the mandrel, the polymer injection system, or combinations or portions thereof. Without wishing to be bound by theory, as the polymer solution is ejected from the polymer injection system, it is thought to be destabilized due to its exposure to a charge. The destabilized solution may then be attracted to a charged mandrel. As the destabilized solution moves from the polymer injection system to the mandrel, its solvents may evaporate and the polymer may stretch, leaving a long, thin fiber that is deposited onto the mandrel. The polymer solution may form a Taylor cone as it is ejected from the polymer injection system and exposed to a charge.

In certain embodiments, a first polymer solution comprising a first polymer and a second polymer solution comprising a second polymer may each be used in a separate polymer injection system at substantially the same time to produce one or more electrospun fibers comprising the first polymer interspersed with one or more electrospun fibers comprising the second polymer. Such a process may be referred to as “co-spinning” or “co-electrospinning,” and a scaffold produced by such a process may be described as a co-spun or co-electrospun scaffold.

### Polymer Injection System

A polymer injection system may include any system configured to eject some amount of a polymer solution into an atmosphere to permit the flow of the polymer solution from the injection system to the mandrel. In some embodiments, the polymer injection system may deliver a continuous or linear stream with a controlled volumetric flow rate of a polymer solution to be formed into a fiber. In some embodiments, the polymer injection system may deliver a variable stream of a polymer solution to be formed into a fiber. In some embodiments, the polymer injection system may be configured to deliver intermittent streams of a polymer solution to be formed into multiple fibers. In some embodiments, the polymer injection system may include a syringe under manual or automated control. In some embodiments, the polymer injection system may include multiple syringes and multiple needles or needle-like components under individual or combined manual or automated control. In some embodiments, a multi-syringe polymer injection system may include multiple syringes and multiple needles or needle-like components, with each syringe containing the same polymer solution. In some embodiments, a multi-syringe polymer injection system may include multiple syringes and multiple needles or needle-like components, with each syringe containing a different polymer solution. In some embodiments, a charge may be applied to the polymer injection system, or to a portion thereof. In some embodiments, a charge may be applied to a needle or needle-like component of the polymer injection system.

In some embodiments, the polymer solution may be ejected from the polymer injection system at a flow rate of less than or equal to about 5 mL/h per needle. In other embodiments, the polymer solution may be ejected from the polymer injection system at a flow rate per needle in a range from about 0.01 mL/h to about 50 mL/h. The flow rate at which the polymer solution is ejected from the polymer injection system per needle may be, in some non-limiting examples, about 0.01 mL/h, about 0.05 mL/h, about 0.1 mL/h, about 0.5 mL/h, about 1 mL/h, about 2 mL/h, about 3 mL/h, about 4 mL/h, about 5 mL/h, about 6 mL/h, about 7 mL/h, about 8 mL/h, about 9 mL/h, about 10 mL/h, about 11 mL/h, about 12 mL/h, about 13 mL/h, about 14 mL/h, about 15 mL/h, about 16 mL/h, about 17 mL/h, about 18 mL/h, about 19 mL/h, about 20 mL/h, about 21 mL/h, about 22 mL/h, about 23 mL/h, about 24 mL/h, about 25 mL/h, about 26 mL/h, about 27 mL/h, about 28 mL/h, about 29 mL/h, about 30 mL/h, about 31 mL/h, about 32 mL/h, about 33 mL/h, about 34 mL/h, about 35 mL/h, about 36 mL/h, about 37 mL/h, about 38 mL/h, about 39 mL/h, about 40 mL/h, about 41 mL/h, about 42 mL/h, about 43 mL/h, about 44 mL/h, about 45 mL/h, about 46 mL/h, about 47 mL/h, about 48 mL/h, about 49 mL/h, about 50 mL/h, or any range between any two of these values, including endpoints.

As the polymer solution travels from the polymer injection system toward the mandrel, the diameter of the resulting fibers may be in the range of about 0.1 μm to about 10 μm. Some non-limiting examples of electrospun fiber diameters may include about 0.1 μm, about 0.2 μm, about 0.25 μm, about 0.5 μm, about 1 μm, about 2 μm, about 5 μm, about 10 μm, about 20 μm, or ranges between any two of these values, including endpoints. In some embodiments, the electrospun fiber diameter may be from about 0.25 μm to about 20 μm.

### Polymer Solution

In some embodiments, the polymer injection system may be filled with a polymer solution. In some embodiments, the polymer solution may comprise one or more polymers. In some embodiments, the polymer solution may be a fluid formed into a polymer liquid by the application of heat. A polymer solution may include, for example, non-resorbable polymers, resorbable polymers, natural polymers, or a combination thereof.

In some embodiments, the polymers may include, for example, polyethylene terephthalate, polyurethane, polyethylene, polyethylene oxide, polyester, polymethylmethacrylate, polyacrylonitrile, silicone, polycarbonate, polyether ketone ketone, polyether ether ketone, polyether imide, polyamide, polystyrene, polyether sulfone, polysulfone, polyvinyl acetate, polytetrafluoroethylene, polyvinylidene fluoride, polycaprolactone, polylactic acid, polyglycolic acid, polylactide-co-glycolide, polylactide-co-caprolactone, polyglycerol sebacate, polydioxanone, polyhydroxybutyrate, poly-4-hydroxybutyrate), trimethylene carbonate, polydiols, polyesters, collagen, gelatin, fibrin, fibronectin, albumin, hyaluronic acid, elastin, chitosan, alginate, silk, copolymers thereof, and combinations thereof.

It may be understood that polymer solutions may also include a combination of one or more of non-resorbable, resorbable polymers, and naturally occurring polymers in any combination or compositional ratio. In an alternative embodiment, the polymer solutions may include a combination of two or more non-resorbable polymers, two or more resorbable polymers or two or more naturally occurring polymers. In some non-limiting examples, the polymer solution may comprise a weight percent ratio of, for example, from about 5% to about 90%. Non-limiting examples of such weight percent ratios may include about 5%, about 10%, about 15%, about 20%, about 25%, about 30%, about 33%, about 35%, about 40%, about 45%, about 50%, about 55%, about 60%, about 66%, about 70%, about 75%, about 80%, about 85%, about 90%, or ranges between any two of these values, including endpoints.

In some embodiments, the polymer solution may comprise one or more solvents. In some embodiments, the solvent may comprise, for example, acetone, dimethylformamide, dimethylsulfoxide, N-methylpyrrolidone, N,N-dimethylformamide, Nacetonitrile, hexanes, ether, dioxane, ethyl acetate, pyridine, toluene, xylene, tetrahydrofuran, trifluoroacetic acid, hexafluoroisopropanol, acetic acid, dimethylacetamide, chloroform, dichloromethane, water, alcohols, ionic compounds, or combinations thereof. The concentration range of polymer or polymers in solvent or solvents may be, without limitation, from about 1 wt % to about 50 wt %. Some non-limiting examples of polymer concentration in solution may include about 1 wt %, 3 wt %, 5 wt %, about 10 wt %, about 15 wt %, about 20 wt %, about 25 wt %, about 30 wt %, about 35 wt %, about 40 wt %, about 45 wt %, about 50 wt %, or ranges between any two of these values, including endpoints.

In some embodiments, the polymer solution and/or the resultant electrospun polymer fiber(s) may also include additional materials. Non-limiting examples of such additional materials may include radiation opaque materials, contrast agents, electrically conductive materials, fluorescent materials, luminescent materials, antibiotics, growth factors, vitamins, cytokines, steroids, anti-inflammatory drugs, small molecules, sugars, salts, peptides, proteins, cell factors, DNA, RNA, other materials to aid in non-invasive imaging, or any combination thereof. In some embodiments, the radiation opaque materials may include, for example, barium, tantalum, tungsten, iodine, gadolinium, gold, platinum, bismuth, or bismuth (III) oxide. In some embodiments, the electrically conductive materials may include, for example, gold, silver, iron, or polyaniline.

In some embodiments, the additional materials may be present in the polymer solution in an amount from about 1 wt % to about 1500 wt % of the polymer mass. In some non-limiting examples, the additional materials may be present in the polymer solution in an amount of about 1 wt %, about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, about 95 wt %, about 100 wt %, about 125 wt %, about 150 wt %, about 175 wt %, about 200 wt %, about 225 wt %, about 250 wt %, about 275 wt %, about 300 wt %, about 325 wt %, about 350 wt %, about 375 wt %, about 400 wt %, about 425 wt %, about 450 wt %, about 475 wt %, about 500 wt %, about 525 wt %, about 550 wt %, about 575 wt %, about 600 wt %, about 625 wt %, about 650 wt %, about 675 wt %, about 700 wt %, about 725 wt %, about 750 wt %, about 775 wt %, about 800 wt %, about 825 wt %, about 850 wt %, about 875 wt %, about 900 wt %, about 925 wt %, about 950 wt %, about 975 wt %, about 1000 wt %, about 1025 wt %, about 1050 wt %, about 1075 wt %, about 1100 wt %, about 1125 wt %, about 1150 wt %, about 1175 wt %, about 1200 wt %, about 1225 wt %, about 1250 wt %, about 1275 wt %, about 1300 wt %, about 1325 wt %, about 1350 wt %, about 1375 wt %, about 1400 wt %, about 1425 wt %, about 1450 wt %, about 1475 wt %, about 1500 wt %, or any range between any of these two values, including endpoints. In one embodiment, the polymer solution may include tantalum present in an amount of about 10 wt % to about 1,500 wt %.

The type of polymer in the polymer solution may determine the characteristics of the electrospun fiber. Some fibers may be composed of polymers that are bio-stable and not absorbable or biodegradable when implanted. Such fibers may remain generally chemically unchanged for the length of time in which they remain implanted. Alternatively, fibers may be composed of polymers that may be absorbed or bio-degraded over time. Such fibers may act as an initial template or scaffold during a healing process. These templates or scaffolds may degrade in vivo once the tissues have a degree of healing by natural structures and cells. It may be further understood that a polymer solution and its resulting electrospun fiber(s) may be composed or more than one type of polymer, and that each polymer therein may have a specific characteristic, such as bio-stability, biodegradability, or bioabsorbability.

### Applying Charges to Electrospinning Components

In an electrospinning system, one or more charges may be applied to one or more components, or portions of components, such as, for example, a mandrel or a polymer injection system, or portions thereof. In some embodiments, a positive charge may be applied to the polymer injection system, or portions thereof. In some embodiments, a negative charge may be applied to the polymer injection system, or portions thereof. In some embodiments, the polymer injection system, or portions thereof, may be grounded. In some embodiments, a positive charge may be applied to mandrel, or portions thereof. In some embodiments, a negative charge may be applied to the mandrel, or portions thereof. In some embodiments, the mandrel, or portions thereof, may be grounded. In some embodiments, one or more components or portions thereof may receive the same charge. In some embodiments, one or more components, or portions thereof, may receive one or more different charges.

The charge applied to any component of the electrospinning system, or portions thereof, may be from about −15 kV to about 30 kV, including endpoints. In some non-limiting examples, the charge applied to any component of the electrospinning system, or portions thereof, may be about −15 kV, about −10 kV, about −5 kV, about −4 kV, about −3 kV, about −1 kV, about −0.01 kV, about 0.01 kV, about 1 kV, about 5 kV, about 10 kV, about 11 kV, about 11.1 kV, about 12 kV, about 15 kV, about 20 kV, about 25 kV, about 30 kV, or any range between any two of these values, including endpoints. In some embodiments, any component of the electrospinning system, or portions thereof, may be grounded.

### Mandrel Movement During Electrospinning

During electrospinning, in some embodiments, the mandrel may move with respect to the polymer injection system. In some embodiments, the polymer injection system may move with respect to the mandrel. The movement of one electrospinning component with respect to another electrospinning component may be, for example, substantially rotational, substantially translational, or any combination thereof. In some embodiments, one or more components of the electrospinning system may move under manual control. In some embodiments, one or more components of the electrospinning system may move under automated control. In some embodiments, the mandrel may be in contact with or mounted upon a support structure that may be moved using one or more motors or motion control systems. The pattern of the electrospun fiber deposited on the mandrel may depend upon the one or more motions of the mandrel with respect to the polymer injection system. In some embodiments, the mandrel surface may be configured to rotate about its long axis. In one non-limiting example, a mandrel having a rotation rate about its long axis that is faster than a translation rate along a linear axis, may result in a nearly helical deposition of an electrospun fiber, forming windings about the mandrel. In another example, a mandrel having a translation rate along a linear axis that is faster than a rotation rate about a rotational axis, may result in a roughly linear deposition of an electrospun fiber along a liner extent of the mandrel.

### Bone Growth Facilitation Device and Methods of Manufacture and Use

The instant disclosure is directed to devices and methods for facilitating bone growth. It may be understood that the devices and methods described herein may be applied to any bone or bone defect, and that the defect examples described herein are non-limiting.

Surgical reconstruction of bone defects is becoming increasingly common, and many conditions may necessitate such reconstruction. Some surgical techniques seek to encourage new bone growth (“osteogenesis”). Encouraging osteogenesis may help provide a fixation point for a permanent implant, or it may be necessary for a defect that is too large to repair with fixation alone. Bone growth facilitation may be particularly important in the context of spinal surgery, where bone grafting can allow osteogenesis to occur across a gap that could otherwise not be bridged by new bone. Without wishing to be bound by theory, the devices and methods disclosed herein may be used to fill a bone defect or a portion thereof, and may encourage osteogenesis therein. The devices disclosed herein include electrospun polymer fibers, which may provide a high surface area interface upon which tissue adhesion, infiltration, and remodeling may be initiated while holding bone graft materials in a desired configuration. The electrospun polymer fibers may also allow for a high degree of flexibility, thus allowing the device to be easily manipulated during placement and usage. The devices may further be porous such that they are highly permeable to blood, cells, and other osteogenic compounds in the body, either endogenous or exogenous, thereby facilitating rapid bone growth. Optionally, such permeability may also facilitate biodegradation or bioresorption of the electrospun polymer fibers.

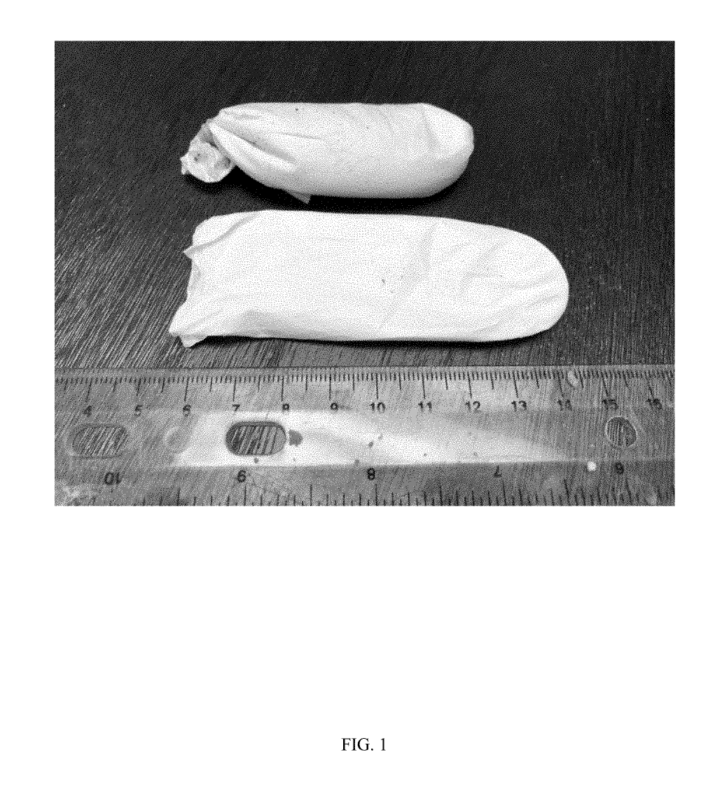

In an embodiment, a device for facilitating bone growth may comprise a porous flexible tube comprising an electrospun fiber. In some embodiments, the porous flexible tube may comprise a closed end. FIG. 1 illustrates embodiments of such a device. In one embodiment, the porous flexible tube may have two closed ends. In certain embodiments, the electrospun polymer fiber may comprise a bioresorbable polymer. In some embodiments, the electrospun polymer fiber may comprise a polymer selected from the group consisting of polyethylene terephthalate, polyurethane, polyethylene, polyethylene oxide, polyester, polymethylmethacrylate, polyacrylonitrile, silicone, polycarbonate, polyether ketone ketone, polyether ether ketone, polyether imide, polyamide, polystyrene, polyether sulfone, polysulfone, polyvinyl acetate, polytetrafluoroethylene, polyvinylidene fluoride, polycaprolactone, polylactic acid, polyglycolic acid, polylactide-co-glycolide, polylactide-co-caprolactone, polyglycerol sebacate, polydioxanone, polyhydroxybutyrate, poly-4-hydroxybutyrate, trimethylene carbonate, polydiols, polyesters, collagen, gelatin, fibrin, fibronectin, albumin, hyaluronic acid, elastin, chitosan, alginate, silk, copolymers thereof, and combinations thereof.

In other embodiments, the electrospun polymer fiber may comprise at least two electrospun polymer fibers, each electrospun polymer fiber comprising a polymer independently selected from the group consisting of polyethylene terephthalate, polyurethane, polyethylene, polyethylene oxide, polyester, polymethylmethacrylate, polyacrylonitrile, silicone, polycarbonate, polyether ketone ketone, polyether ether ketone, polyether imide, polyamide, polystyrene, polyether sulfone, polysulfone, polyvinyl acetate, polytetrafluoroethylene, polyvinylidene fluoride, polycaprolactone, polylactic acid, polyglycolic acid, polylactide-co-glycolide, polylactide-co-caprolactone, polyglycerol sebacate, polydioxanone, polyhydroxybutyrate, poly-4-hydroxybutyrate, trimethylene carbonate, polydiols, polyesters, collagen, gelatin, fibrin, fibronectin, albumin, hyaluronic acid, elastin, chitosan, alginate, silk, copolymers thereof, and combinations thereof, and wherein the at least two electrospun polymer fibers are co-electrospun. In certain embodiments, the electrospun polymer fiber comprises co-electrospun polycaprolactone (PCL) fibers and polylactide-co-caprolactone (PLCL) fibers. In other embodiments, the electrospun polymer fiber comprises co-electrospun polycaprolactone fibers and polystyrene fibers.

In certain embodiments, the porous flexible tube may comprise at least two electrospun polymer fibers. In one embodiment, the electrospun polymer fibers may be randomly oriented with respect to one another. In another embodiment, the electrospun polymer fibers may be aligned or substantially aligned with respect to one another, such that the fibers are substantially parallel to one another. In still other embodiments, the electrospun polymer fibers may be a combination of randomly oriented and aligned or substantially aligned with respect to one another.

In some embodiments, the porous flexible tube may comprise pores having a diameter of about 5 μm to about 50 μm. In certain embodiments, the pores may be homogenously distributed or substantially homogenously distributed throughout the porous flexible tube, while in other embodiments, the pores may be concentrated at an end or at the center of the porous flexible tube. In some embodiments, the pores may have a diameter or an average diameter of, for example, about 5 μm, about 10 μm, about 15 μm, about 20 μm, about 25 μm, about 30 μm, about 35 μm, about 40 μm, about 45 μm, about 50 μm, or any range between any two of these values, including endpoints.

In some embodiments, the porous flexible tube may comprise an inner diameter of about 5 mm to about 20 mm. The inner diameter may be, for example, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, about 20 mm, or any range between any two of these values, including endpoints.

In some embodiments, the porous flexible tube may comprise a length of about 1 cm to about 20 cm. The length of the porous flexible tube may be, for example, about 1 cm, about 2 cm, about 3 cm, about 4 cm, about 5 cm, about 6 cm, about 7 cm, about 8 cm, about 9 cm, about 10 cm, about 11 cm, about 12 cm, about 13 cm, about 14 cm, about 15 cm, about 16 cm, about 17 cm, about 18 cm, about 19 cm, about 20 cm, or any range between any two of these values, including endpoints.

In certain embodiments, the device may further comprise a filler material at least partially encased by the porous flexible tube. In some embodiments, the filler material may be fully encased by the porous flexible tube. In an embodiment, the filler material may be selected from the group consisting of autologous bone, allogeneic bone, human cadaver bone, demineralized bone, xenograft bone, hydroxyapatite, tricalcium phosphate, bioactive glass, a growth factor, particles thereof, and combinations thereof.

In some embodiments, a method of manufacturing a device for facilitating bone growth as described herein may comprise electrospinning a polymer solution onto an end of a cylindrical mandrel to form a porous flexible tube comprising an electrospun polymer fiber, and removing the porous flexible tube from the end of the cylindrical mandrel. In certain embodiments, the porous flexible tube may comprise a closed end. In an embodiment, a method of manufacturing a device for facilitating bone growth as described herein may comprise electrospinning a polymer solution onto an end of a cylindrical mandrel to form a porous flexible tube comprising an electrospun polymer fiber and having a closed end, and removing the porous flexible tube from the end of the cylindrical mandrel. In certain embodiments, the mandrel may be partially or substantially cylindrical. In other embodiments, the mandrel may be fully cylindrical.

In certain embodiments, the step of electrospinning may form a porous flexible tube comprising an electrospun polymer fiber and having two closed ends. In an embodiment, the method further comprises at least partially encasing a filler material as described herein within the porous flexible tube.

In some embodiments, the electrospun polymer fiber may be any electrospun polymer fiber as described herein. In certain embodiments, the step of electrospinning comprise co-electrospinning a first polymer solution and a second polymer solution. In one non-limiting example, the first polymer solution may comprise polycaprolactone and the second polymer solution may comprise polylactide-co-caprolactone.

In some embodiments, the step of electrospinning may create pores by including spaces between the one or more electrospun polymer fibers. In other embodiments, the method of manufacturing may further comprise adding pores to the porous flexible tube. In certain embodiments, pores may be added using a laser ablation technique. In other embodiments, pores may be added by any technique known in the art.

In some embodiments, a method of facilitating bone growth may comprise obtaining a porous flexible tube as described herein, and implanting the porous flexible tube into a bone defect of a subject. In other embodiments, the porous flexible tube may be implanted into another region of a subject. In certain embodiments, the method may further comprise at least partially encasing a filler material within the porous flexible tube, as described herein.

In certain embodiments, implanting the porous flexible tube into the bone defect of the subject may comprise affixing the porous flexible tube to at least a portion of the defect. The fixation may be accomplished, for example, using a surgical fixation device such as a suture, a screw, a pin, a plate, or a combination thereof; the fixation may also be accomplished using any surgically acceptable adhesive. In other embodiments, implanting the porous flexible tube into the bone defect of the subject may comprise simply placing the porous flexible tube in contact with at least a portion of the bone defect.

### EXAMPLES

### Example 1

In one example, bone ingrowth, local tissue response, and biomechanical effectiveness of repair of the rotator cuff with embodiments of electrospun polymer fibers described herein were studied in an ovine (sheep) model. Forty animals were divided into two groups: a treatment group (20 animals), which was treated with electrospun polymer fibers as described herein, and a control group (20 animals), which underwent rotator cuff repairs, but did not receive electrospun polymer fibers. The contralateral untreated shoulders of 10 animals were collected and used as untreated samples.

In the treatment and control groups, the right infraspinatus tendon was completely transected at the humeral attachment and acutely reattached to the humeral footprint using a total of four suture anchors. In the treatment group, electrospun polymer fibers were sandwiched between the infraspinatus tendon and humeral footprint. Ten animals from each group were sacrificed 6 weeks after the repair, and the remaining ten animals from each group were sacrificed 12 weeks after the repair.

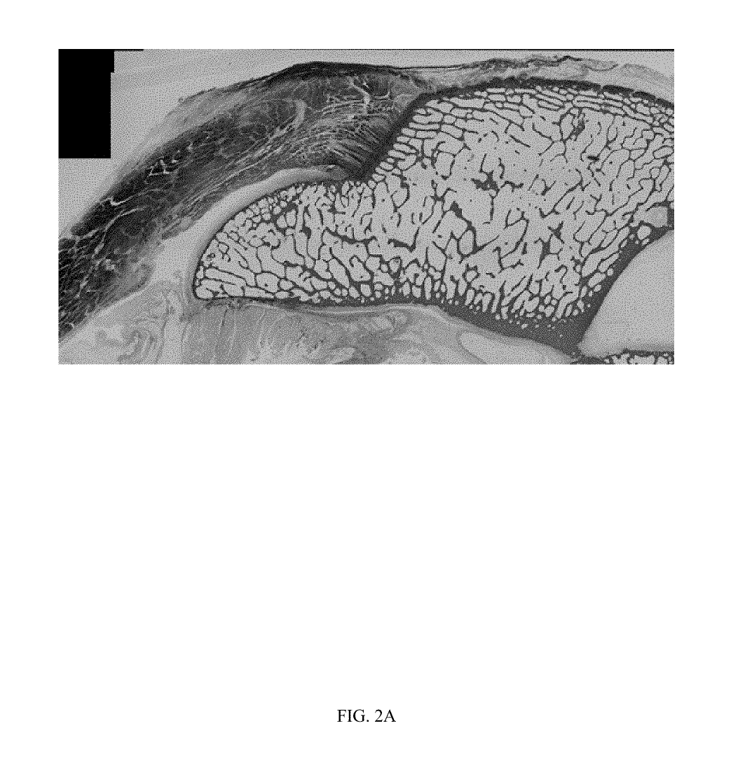

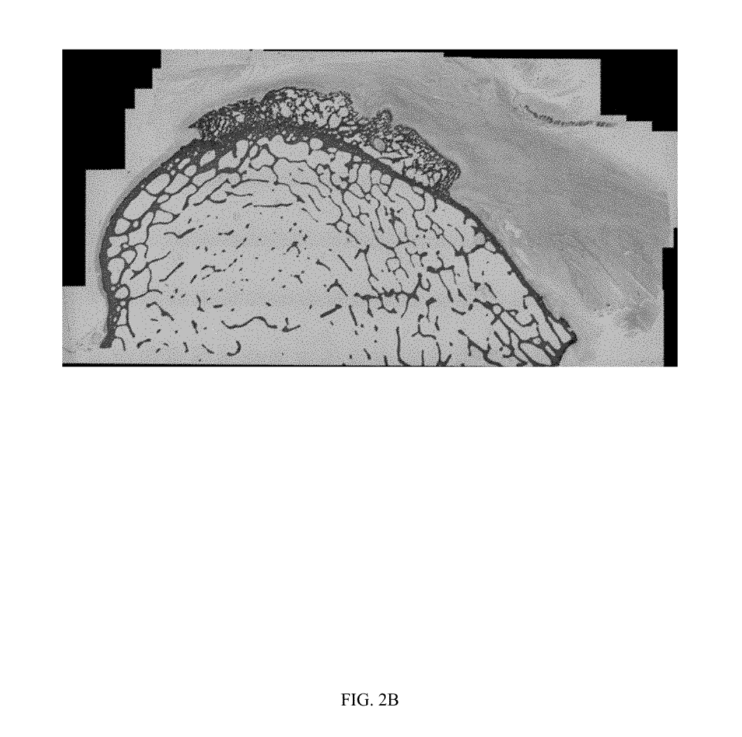

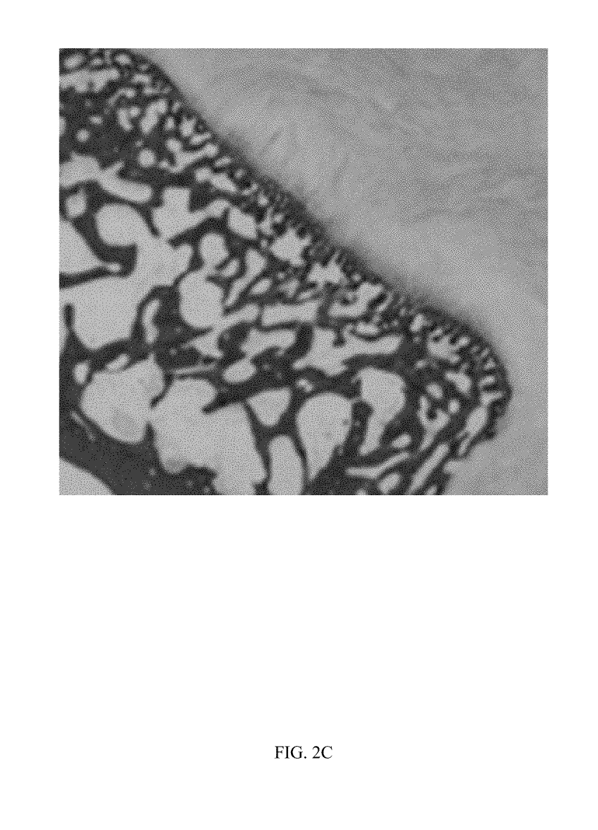

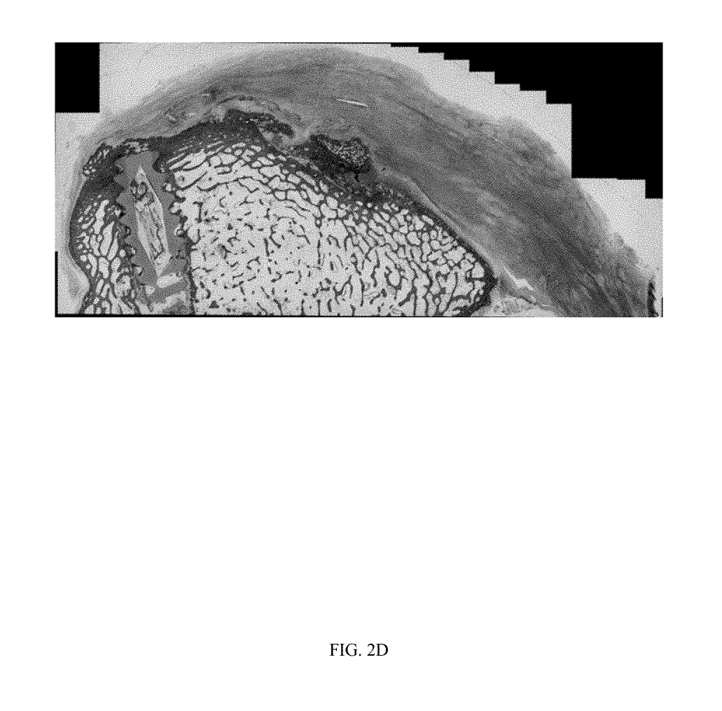

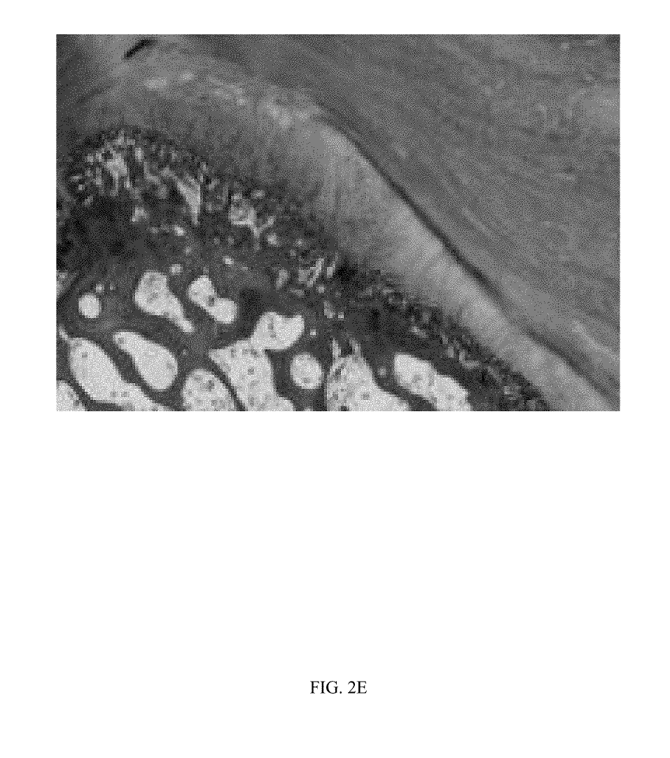

FIG. 2A illustrates a histological sample of an untreated control rotator cuff (i.e. a contralateral control). FIG. 2B illustrates a histological sample of a treated control rotator cuff at 12 weeks (i.e. a rotator cuff that underwent a repair but did not receive electrospun polymer fibers). FIG. 2C illustrates a magnified view of a portion of the histological sample shown in FIG. 2B. FIG. 2D illustrates a histological sample of a treated rotator cuff at 12 weeks (i.e. a rotator cuff that underwent a repair and received electrospun polymer fibers as described herein). FIG. 2E illustrates a magnified view of a portion of the histological sample shown in FIG. 2D. This histology illustrates a fibrous scar between the bone and tendon with the suture repair in the samples repaired without electrospun polymer fibers (e.g., FIG. 2B and FIG. 2C). Such a scar was not present in the samples repaired with electrospun polymer fibers as described herein (e.g. FIG. 2D and FIG. 2E).

### Example 2

In another example, bone ingrowth, local tissue response, and biomechanical effectiveness of cartilage repair with embodiments of electrospun polymer fibers (specifically, co-electrospun PLCL+PGA scaffold, referred to below as the “Nanofiber Solutions scaffold”) described herein, as compared to a commercially available allograft product (CARTIFORM® Viable Osteochondral Allograft, referred to below as the “Cartiform” scaffold) were studied in a rabbit model (6 rabbits; N=12 defects).

### Experimental Design and Procedures

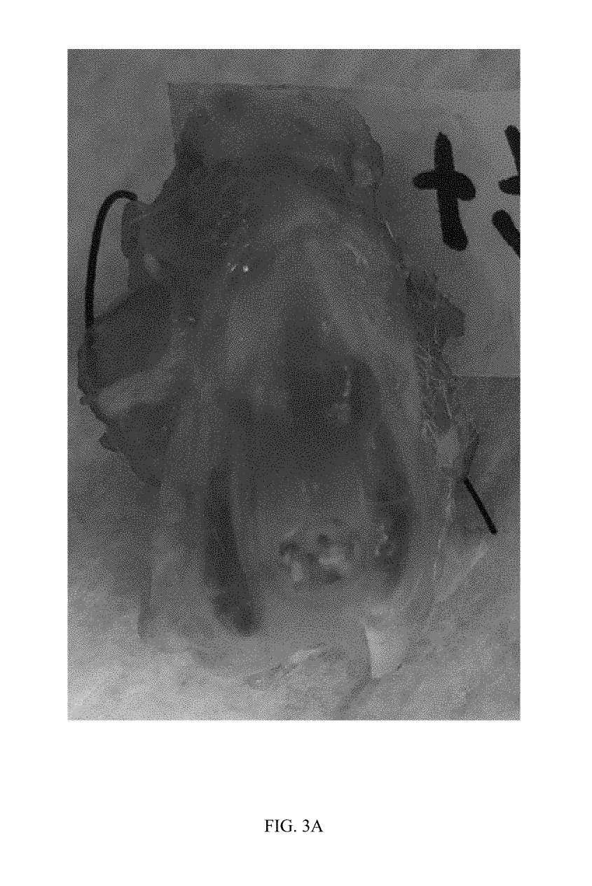

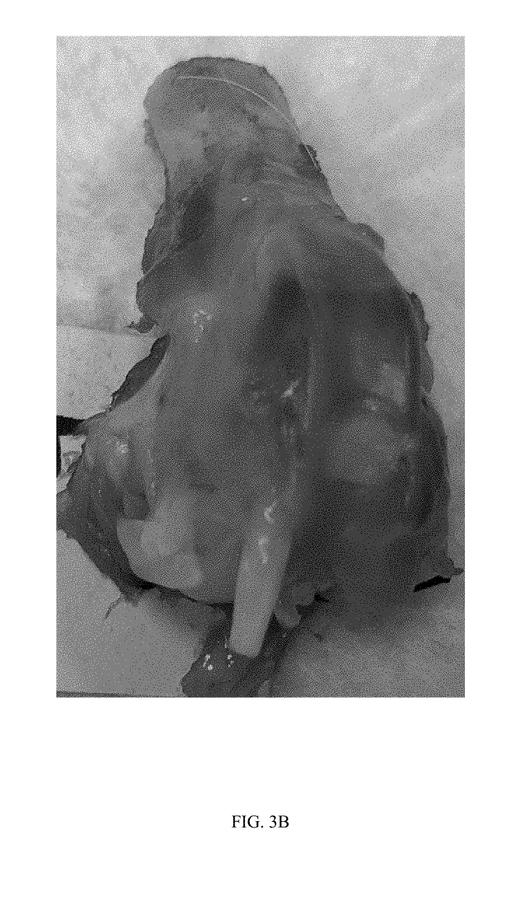

Each rabbit was anesthetized and underwent creation of two defects on the distal surface of the femurs. Briefly, lateral parapatellar skin incisions were made and the patellas were dislocated laterally. Afterwards, the knees were flexed to expose the medial femoral condyles. A defect was created (approximately 4.75 mm in diameter and 4 mm deep) extending through the cartilage into the subchondral bone. Following creation of the defect, each defect was assigned to receive one of two possible treatments:
1. Cartiform (See FIG. 3A)
2. Nanofiber scaffold (See FIG. 3B)

The Nanofiber Solutions scaffold was cut into a 4.75 mm diameter disc to match the defect size prior to implant/mixing with other substrates for implant. Following creation and repair of the defect, the rabbit was allowed to recover for up to 6 weeks. Following recovery, rabbits were euthanized and graft tissues harvested for histopathology assessment.

### Histology

Histology was performed on 6 rabbits (N=12 defects). At necropsy, gross dissection (i.e., limbs isolated, skin removed) was completed, after which samples were placed in 10% neutral buffered formalin.

Following gross dissection and fixation (a minimum of two (N=2) weeks in formalin), surgical defect sites were isolated by creating a slab of tissue approximately 1 cm thik in the sagittal plane. Images were taken of the gross tissue blocks. After fixation, the tissue was dehydrated in graded solutions of ETOH on a tissue processor (Tissue-Tek VIP, Sakura, Torrance, Calif.). After processing, the samples were cleared with acetone and polymerized into a hardened plastic block using Hard Acrylosin (Dorn and Hart Microedge).

Histological sections were taken in the sagittal plane to display the defect site, articulating surface, and surrounding bone. One (N=1) slide was cut through each ROI. Initial sections were taken using an Exakt diamond blade bone saw at a thickness of approximately 300-400 μm. All sections were ground using an Exakt microgrinder to 60-70 μm thickness and stained. Sections were first stained with Sanderson's Rapid Bone stain, which provides differentiation of cells within the section and allows detection of cartilage within the tissue. Slides were then counterstained using a Van Gieson bone stain that allows differentiation of collagen and detection of bone (immature woven bone and mature lamellar bone) within the section. A total of 12 slides were produced from 6 animals.

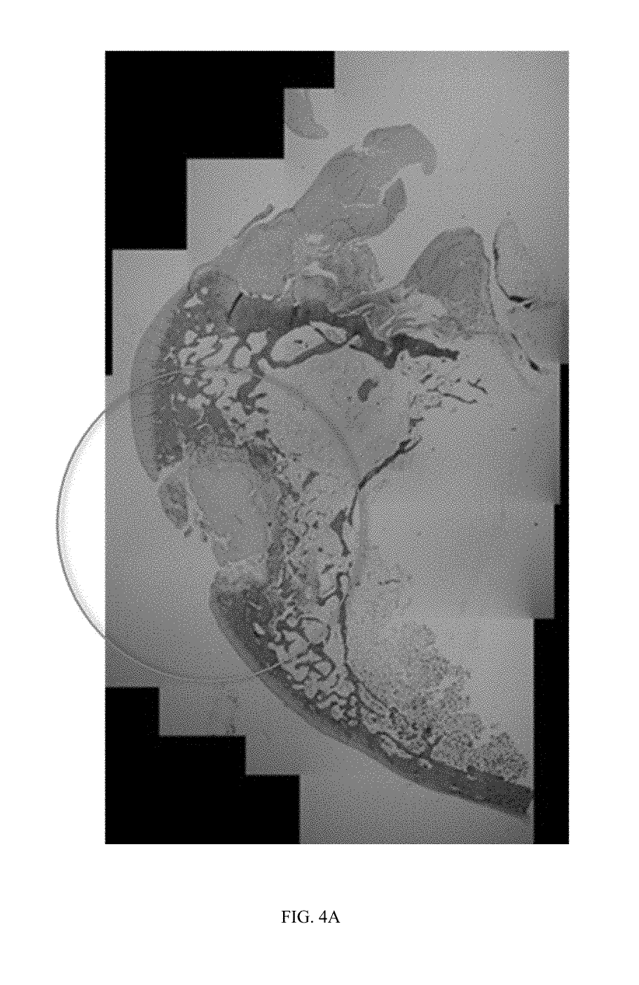

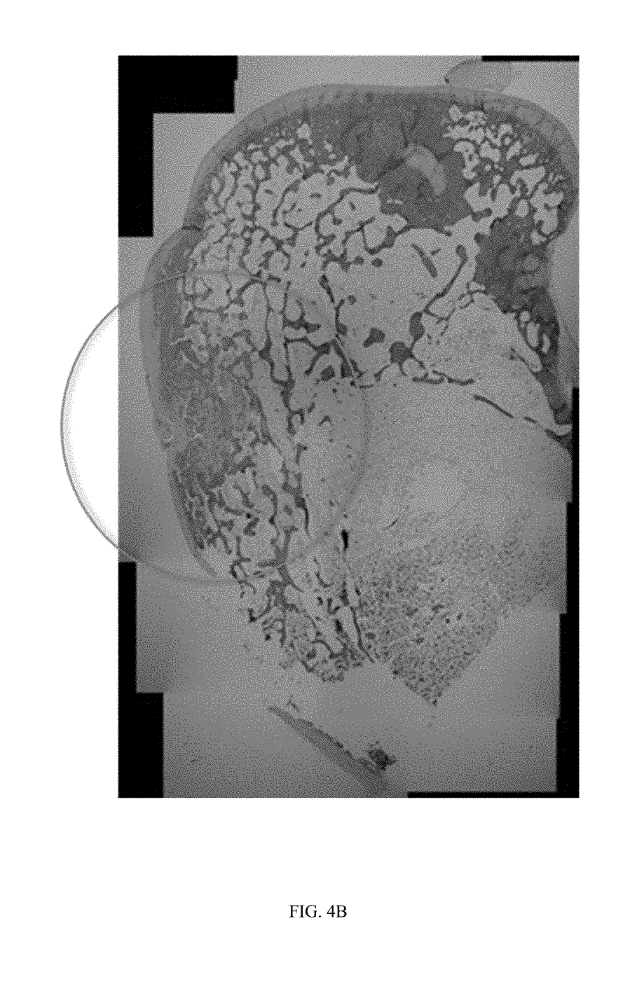

High-resolution digital images were acquired by field for the all surgical site slides using a Nikon E800 microscope (AG Heinze, Lake Forest, Calif.), Spot digital camera (Diagnostic Instruments, Sterling, Heights, Mich.), a Pentium IBM-based computer with expanded memory capabilities (Dell Computer Corp., Round Rock, Tex.). See FIG. 4A and FIG. 4B.

### Results

Table 1 below indicates the scores of the blinded DVM scoring of histology sections.

TABLE 1
Blinded DVM Scoring

| Animal ID | Type of Repair | Cartilage Defect Repair Scores |
|---|---|---|
| 215L | Nanofiber scaffold | 0 |
| | | 0 |
| 215R | Cartiform ® | 3 |
| | | 3 |
| 217L | Cartiform ® | 0 |
| | | 0 |
| 217R | Nanofiber scaffold | 1 |
| | | 2 |
| 218L | Nanofiber scaffold | 2 |
| | | 2 |
| 218R | Cartiform ® | 3 |
| | | 3 |
| 219L | Cartiform ® | 0 |
| | | 0 |
| 219R | Nanofiber scaffold | 4 |
| | | 4 |
| 220L | Nanofiber scaffold | 1 |
| | | 1 |
| 220R | Cartiform ® | 1 |
| | | 1 |
| 221L | Cartiform ® | 1 |
| | | 1 |
| 221R | Nanofiber scaffold | 1 |
| | | 2 |

| | |
|---|---|
| Cartiform ® Avg. | 1.33 |
| Nanofiber Scaffold Avg. | 1.67 |

Animal 215L had a very large defect created by accident during implantation and should be removed from the analyses. Doing so would increase the Nanofiber Scaffold average score to 2.00.

FIG. 4A shows the histological sample of the femur repaired by Cartiform. Centrally, this defect is filled with a large devitalized fragment of hyaline-like cartilage (allograft). Circumferentially surrounding this allograft, lining the margin of the defect, and extending into and filling the adjacent medullary spaces is moderate amounts of dense fibrous connective tissue. There is no histological evidence of re-establishment of an articular cartilage surface.

FIG. 4B shows the histological sample of the rabbit femur repaired using a Nanofiber Solutions scaffold per embodiments of the disclosure herein. As shown by the figure, the defect is filled with tissue and appears well-integrated into the surrounding host bone. Completely filling the defect is an approximately 50/50 mixture composed of trabeculae of new woven bone or hyaline-like cartilage.

Diffusely, the superficial surface of the defect is completely filled with dense hyaline cartilage. This cartilage is disorganized and lacks normal hyaline-structure of chondrocytes but is re-establishing the normal articular surface.

While the present disclosure has been illustrated by the description of exemplary embodiments thereof, and while the embodiments have been described in certain detail, it is not the intention of the Applicants to restrict or in any way limit the scope of the appended claims to such detail. Additional advantages and modifications will readily appear to those skilled in the art. Therefore, the disclosure in its broader aspects is not limited to any of the specific details, representative devices and methods, and/or illustrative examples shown and described. Accordingly, departures may be made from such details without departing from the spirit or scope of the Applicant's general inventive concept.

1. A device for facilitating bone growth, the device comprising:
a porous flexible tube comprising an electrospun polymer fiber.

2. The device of claim 1, wherein the porous flexible tube comprises at least one closed end.

3. The device of claim 2, further comprising a filler material at least partially encased by the porous flexible tube.

4. The device of claim 3, wherein the filler material comprises a bone graft material selected from the group consisting of autologous bone, allogeneic bone, human cadaver bone, demineralized bone, xenograft bone, hydroxyapatite, tricalcium phosphate, bioactive glass, a growth factor, particles thereof, and combinations thereof.

5. The device of claim 1, wherein the electrospun polymer fiber comprises co-electrospun fibers selected from the group consisting of polyglycolide fibers, polycaprolactone fibers, and polylactide-co-caprolactone fibers.

6. The device of claim 1, wherein the electrospun polymer fiber further comprises an additional material selected from the group consisting of an antibiotic, a growth factor, a vitamin, a cytokine, a protein, and combinations thereof.

7. The device of claim 1, wherein the porous flexible tube comprises pores having a diameter of about 5 μm to about 50 μm, and wherein the porous flexible tube comprises an inner diameter of about 5 mm to about 20 mm.

8. A method of manufacturing a device for facilitating bone growth, the method comprising:
electrospinning a polymer solution onto a cylindrical mandrel to form a porous flexible tube comprising an electrospun polymer fiber; and
removing the porous flexible tube from the cylindrical mandrel.

9. The method of claim 8, wherein electrospinning comprises electrospinning the polymer solution onto an end of the cylindrical mandrel, and wherein the porous flexible tube comprises at least one closed end.

10. The method of claim 9, further comprising at least partially encasing a filler material within the porous flexible tube.

11. The method of claim 10, wherein the filler material comprises a bone graft material selected from the group consisting of autologous bone, allogeneic bone, human cadaver bone, demineralized bone, xenograft bone, hydroxyapatite, tricalcium phosphate, bioactive glass, a growth factor, particles thereof, and combinations thereof.

12. The method of claim 8, wherein the electrospun polymer fiber further comprises an additional material selected from the group consisting of an antibiotic, a growth factor, a vitamin, a cytokine, a protein, and combinations thereof.

13. The method of claim 8, wherein the porous flexible tube comprises pores having a diameter of about 5 μm to about 50 μm, and wherein the porous flexible tube comprises an inner diameter of about 5 mm to about 20 mm.

14. A method of facilitating bone growth, the method comprising:
obtaining a porous flexible tube comprising an electrospun polymer fiber; and
implanting the porous flexible tube into a bone defect of a subject.

15. The method of claim 14, wherein the porous flexible tube comprises at least one closed end.

16. The method of claim 15, further comprising at least partially encasing a filler material within the porous flexible tube.

17. The method of claim 16, wherein the filler material comprises a bone graft material selected from the group consisting of autologous bone, allogeneic bone, human cadaver bone, demineralized bone, xenograft bone, hydroxyapatite, tricalcium phosphate, bioactive glass, a growth factor, particles thereof, and combinations thereof.

18. The method of claim 14, wherein the electrospun polymer fiber further comprises an additional material selected from the group consisting of an antibiotic, a growth factor, a vitamin, a cytokine, a protein, and combinations thereof.

19. The method of claim 14, wherein the porous flexible tube comprises pores having a diameter of about 5 μm to about 50 μm, and wherein the porous flexible tube comprises an inner diameter of about 5 mm to about 20 mm.

20. The method of claim 14, wherein implanting the porous flexible tube into the bone defect of the subject comprises placing the porous flexible tube in contact with a portion of the bone defect.
